(19) United States
(12) Patent Application Publication
(10) Pub. No.: US 2008/0119751 A1
(43) Pub. Date: May 22, 2008

(54) CALORIMETRY

(75) Inventor: Michael Flanagan, Manchester (GB)

Correspondence Address: HUSCH BLACKWELL SANDERS LLP, 720 OLIVE STREET, SUITE 2400, ST. LOUIS, MO, 63101, US

(73) Assignee: NUTREN TECHNOLOGY LIMITED, Lancashire (GB)

(21) Appl. No.: 11/611,131

(22) Filed: Dec. 14, 2006

(30) Foreign Application Priority Data
Oct. 11, 2006 (GB) 0620082.8

(51) Int. Cl. A61B 5/08

(52) U.S. Cl. 600/531

(57) ABSTRACT

A calorimeter (1-7) comprising a volume sensor (11) to measure a volume of a breath, and a processing means (13) to calculate a metabolic rate from the output of the volume sensor; and wherein the calorimeter calculates a metabolic rate without means to measure the chemical constituents present in a breath.

### CROSS-REFERENCE TO RELATED APPLICATION

This application claims the benefit of United Kingdom Application No. 0620082.8, filed on Oct. 11, 2006 in the United Kingdom Intellectual Property Office, the disclosure of which is incorporated herein by reference.

### FIELD OF THE INVENTION

The present invention relates to calorimeters and methods for processing calorimetric data.

### BACKGROUND TO THE INVENTION

Calorimetric techniques are used to study the energy of metabolism in humans and animals. The techniques can be used, for example, by sport or nutritional scientists for the diagnosis of metabolic disorders and for calculating nutritional requirements. The techniques can be either direct or indirect. Direct techniques involve measuring the heat loss of a subject. Indirect techniques involve measuring the chemical by-products of metabolism.

Indirect calorimetry involves calculating the energy produced by a subject by measuring their carbon dioxide production or oxygen consumption. Such measurements can be done in a number of ways. However, the indirect calorimeter considered to be the “gold standard” or most accurate is the Douglas Bag.

A common technique for using the carbon dioxide production or oxygen content measurements is to determine a subject's metabolic rate using the Weir equation:

Calories=(3.941*VO2+1.106*RQ*VO2)*(1−0.082*Pf)

Where,
Calories=calories burned per litre of oxygen consumed
   RQ=Respiratory Quotient=VCO2/VO2
VO2=volume of oxygen per unit time
VCO2=volume of carbon dioxide produced per unit time
Pf=the fraction of total energy due to protein oxidation

To obtain the values for carbon dioxide production or oxygen consumption indirect calorimeters use devices such as oxygen sensors, capnometers, flow sensors, etc. The problem with such devices is that they are complicated to manufacture, calibrate and use.

It is an aim of the preferred embodiments of the present invention to provide a calorimeter that is easy to manufacture and use.

### SUMMARY OF THE INVENTION

According to the present invention in a first aspect, there is provided a calorimeter comprising:
a volume sensor to measure a volume of a breath, and
a processing means to calculate a metabolic rate from the output of the volume sensor; and wherein
the calorimeter calculates a metabolic rate without means to measure the chemical constituents present in a breath.

Preferably, the processing means uses a constant OC representing the oxygen content of a breath to calculate a metabolic rate. Preferably, OC is in a range of 6 to 8. Suitably, OC is 6.961.

It has surprisingly been found that the oxygen content of an exhaled breath can be treated as a constant. This constant oxygen content can, using the Weir equation, be used by a calorimeter to calculate the calories used by a user. The constant is different for different calorimeter component configurations and can be determined in comparison with the results of a Douglas Bag. Graph 1 (below) shows the correlation of a calorimeter using the constant with the Douglas Bag. As shown by graph 1 a calorimeter using the constant will determine a metabolic rate measurement with sufficient accuracy to be used by, for example, a home user or a gym user.

Preferably, the processing means is configured to calculate a metabolic rate by multiplying OC by the volume of a breath.

Preferably, the calorimeter determines a metabolic rate only using an output from the volume sensor.

Preferably, the volume sensor measures the volume of at least one breath. Preferably, the processing means calculates a metabolic rate using the volume of at least one breath.

Preferably, the volume sensor measures a volume of a plurality of breaths. Preferably, the processing means calculates a metabolic rate by averaging a volume of a plurality of breaths.

Preferably, the calorimeter is configured to determine a metabolic rate using at least one reference breath.

Preferably, the volume sensor is a pressure sensor. Alternatively, the volume sensor is a flow sensor.

Preferably, the calorimeter comprises a housing. Preferably, the volume sensor is located in the housing.

Preferably, the housing comprises a fluid inlet arranged in fluid communication with the volume sensor. Preferably, the fluid inlet is an opening on the housing. Preferably, the fluid inlet is a tube extending from the housing. Preferably, the tube is inflexible. Alternatively, the tube is flexible. Preferably, the tube is a hose.

Preferably, the fluid inlet comprises a mouthpiece. Preferably, the mouthpiece is separable from the fluid inlet.

Preferably, the calorimeter further comprises a breath counter. Preferably, the breath counter communicates a breath count to the processing means. Preferably, the processing means uses the breath count in the metabolic rate calculation. Preferably, the breath counter is configured to use the volume sensor. Alternatively, the breath counter uses a breath count sensor.

Preferably, the calorimeter further comprises a timing means. Preferably, the timing means communicates a breath time to the processing means.

Preferably, the breath time is used in the metabolic rate calculation. Preferably, the breath time is used when calculating the volume of a breath.

Preferably, the timing means is located in the housing.

Preferably, the calorimeter further comprises an input means to receive at least one user parameter. Preferably, the input means communicates the at least on user parameter to the processing means. Preferably, the at least one user parameter is used in the metabolic rate calculation. Preferably, the input means is located on the housing.

Preferably, the calorimeter further comprises a display to display at least a metabolic rate. Preferably, the display is located on the housing.

Preferably, the calorimeter further comprises a warning means to provide a warning signal to the user. Preferably, the warning signal is provided to indicate a reference breath period has ended. Preferably, the warning signal is provided to indicate that a required number of breaths have been received.

Preferably, the calorimeter further comprises a storage means. Preferably, the storage means stores the volume sensor measurement. Preferably, the storage means is configured to maintain the stored volume sensor measurement until required.

Preferably, the storage means stores a user configuration. Preferably, the storage means stores a plurality of user configurations. Preferably, the calorimeter can be configured based on an individual user configuration retrieved from the plurality of user configurations.

According to the present invention in a second aspect there is provided a method of calculating a metabolic rate, the method comprising the steps of:

(a) providing a calorimeter comprising a volume sensor and a processing means,

(b) receiving at least one breath from a user,

(c) determining the volume of the at least one breath,

(d) calculating the metabolic rate of the user without measuring the chemical constituents present in a breath.

Preferably, step (d) comprises calculating the metabolic rate using a constant OC representing the oxygen content of a breath. Preferably, step (d) comprises calculating the metabolic rate by multiplying the volume of at least one breath by OC.

Preferably, step (a) further comprises providing a timing means and step (c) further comprises providing a breath time and calculating the volume of at least one breath using the breath time.

Preferably, step (a) further comprises providing a breath counter and step (c) further comprises determining the volume of a plurality of breaths. Preferably, step (c) comprises calculating the average volume of the plurality of breaths.

Preferably, step (c) comprises receiving a breath count before calculating the average volume of the plurality of breaths.

Preferably, step (c) comprises receiving a plurality of breathing times before calculating the average volume of the plurality of breaths.

Preferably, the method further comprises step (e) displaying the metabolic rate.

### BRIEF DESCRIPTION OF THE DRAWINGS

For a better understanding of the invention, and to show how embodiments of the same may be carried into effect, reference will now be made, by way of example, to the accompanying diagrammatic drawings in which:

FIG. 1 shows a schematic side view of a first embodiment of the calorimeter of the present invention;

FIG. 2 shows a schematic side view of a second embodiment of the calorimeter of the present invention;

FIG. 3 shows a schematic side view of a third embodiment of the calorimeter of the present invention;

FIG. 4 shows a schematic side view of a fourth embodiment of the calorimeter of the present invention;

FIG. 5 shows a schematic side view of a fifth embodiment of the calorimeter of the present invention;

FIG. 6 shows a schematic side view of a sixth embodiment of the calorimeter of the present invention; and

FIG. 7 shows a schematic side view of a seventh embodiment of the calorimeter of the present invention.

### DETAILED DESCRIPTION OF THE PREFERRED EMBODIMENTS

FIGS. 1 through 7 show the preferred embodiments of the present invention. The preferred embodiments each provide a calorimeter 1-7 for determining the metabolic rate of a user that is less complex to manufacture and easier to calibrate and use.

Each calorimeter 1-7 is an indirect calorimeter. Each calorimeter 1-7 determines the metabolic rate of a user without using means to means to measure the chemical constituents present in a user's breath, i.e., the oxygen content, carbon dioxide content, etc., of a user's breath. Each calorimeter 1-7 determines the metabolic rate of a user from the total volume of a user's breath.

FIG. 1 shows a first embodiment of the calorimeter 1. The calorimeter 1 comprises a housing 10, a volume sensor 11 and a fluid inlet 12 arranged in fluid communication with the volume sensor 11. The calorimeter 1 further comprises a processing means 13.

The calorimeter 1 uses the processing means 13 to calculate the metabolic rate of a user using only the output from the volume sensor 11. In this sense, the calorimeter 1 does not require means to measure the chemical constituents present in a breath or the percentage of chemical constituents present in a breath. The calorimeter 1 determines the metabolic rate of a user without a oxygen sensor, capnometer, nitrogen sensor, and similar devices.

The volume sensor 11 measures inhaled and/or exhaled breath. The volume sensor 11 can be any suitable device capable of producing a measurement suitable for the calculation of a volume of a breath. The volume sensor 11 measures the total volume of a user's breath.

The volume sensor 11 can be a flow sensor. The flow sensor measures amount of flow through the fluid inlet per unit time. The flow measurement can, for example, be used to calculate the velocity of a breath. The velocity along with the known area of the fluid inlet 12 is then used to determine the volume of a breath.

Alternatively, the volume sensor 11 can be a pressure sensor. The pressure sensor measures the pressure exerted by a user's breath. The pressure measurement is then used to calculate the volume of a breath using, for example, the ideal gas equations.

The volume sensor 11 is connected to communicate with the processing means 13. The volume sensor 11 communicates the volume of a breath to the processing means 13 which calculates a user's metabolic rate based on the following equation:

Calories=Constant OC*Volume of measured breath eq. (1)

The constant OC used in eq. 1 is in the range of 6-8 and is determined for the calorimeter 1 based upon the components used. For example, in a calorimeter comprising a Honeywell 20PCSMT pressure sensor used in calculating the volume of a user's breath the value of the constant has been determined to be 6.961.

Graph 1 shows the correlation of the metabolic rate measurements of a Douglas Bag with a calorimeter using the constant and comprising a Honeywell 20PCSMT. The Douglas Bag is considered to be the most accurate indirect calorimeter. As shown by graph 1, the calorimeter 1, using the constant and only the volume of a user's breath, will determine a metabolic rate measurement with an accuracy or R² when compared to the Douglas Bag of 0.82. This accuracy is sufficient for casual use, i.e., a home user, a gym user, etc.

The calorimeter 1 is provided only with a volume sensor 11 to enable the metabolic rate of a user to be determined. The calorimeter 1 can determine the metabolic rate of user without requiring any data other than the volume of a user's breath. For example, the calorimeter 1 can configured to operate with using a oxygen sensor, carbon dioxide sensor, nitrogen sensor, pulse meter, heart rate monitor, and other similar devices.

The calorimeter 1 is configured to measure a metabolic rate based on a single breath and the processing means 13 provides a user with a fast measurement from the signal of the volume sensor 11.

Alternatively, the calorimeter 1 is configured to measure a metabolic rate based on a plurality of breaths or a reference breath and the processing means 13 averages the signals from the volume sensor 11 to provide a user with a measurement of their metabolic rate.

A reference breath can be established through at least one continuous period of breathing. Such a period is, for example, five minutes in duration. The reference breath is an average of the volume of breath sensed during the continuous period of breathing. The reference breath should be taken in a rested state, preferably in the morning after a period of rest.

The accuracy of the calorimeter can be improved using a reference breath. The reference breath enables a more accurate determination of a volume of a user's breath after exercise, during rest, etc.

The fluid inlet 12 is any suitable means capable of communicating a user's breath to the volume sensor 11. The fluid inlet 12 shown in FIG. 1 is a tube. The tube is rigid.

The tube can of course be flexible, for example, a flexible hose. The flexible hose can be any suitable length, for example, the hose can between 5 cm to 100 cm in length.

The calorimeter also comprises a mouthpiece (not shown). The mouthpiece is connected to the fluid inlet 12 at the end 14 of the fluid inlet opposed to the volume sensor 11. The mouthpiece is formed integrally with the fluid inlet 12. Alternatively, the mouth piece can be separable from the fluid inlet 12 to, for example, enable the mouthpiece to be changed for different users. The mouthpiece can also be a mask.

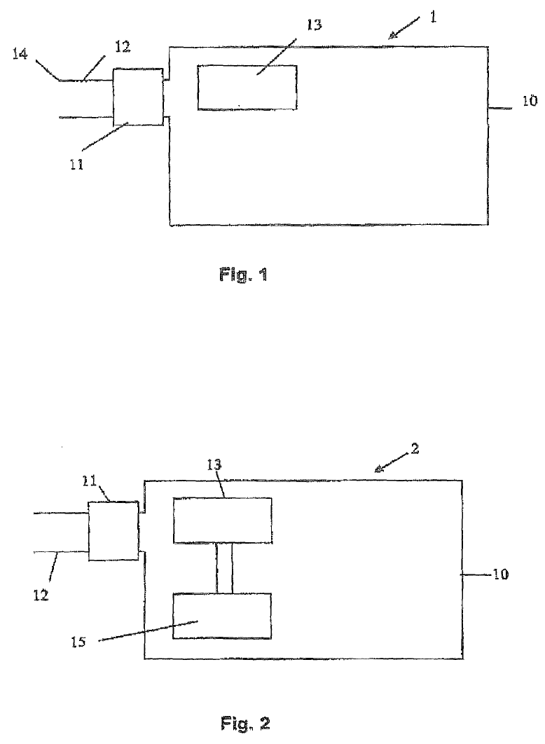

FIG. 2 shows a second embodiment of the calorimeter 2. The calorimeter 2 comprises the features of the first embodiment and a storage means 15 to store the signals from the volume sensor 11. The processing means 13 uses the stored values to calculate the metabolic rate.

The storage means 15 can be used to store the volume sensor 11 measurements in such a way that they are accessible after use of the device. For example, if the processing means 13 is provided in an external apparatus such as a computer, the processing means can access the stored volume sensor measurements when the device is connected to the computer.

The calorimeter 2 is used by a single user. However, the calorimeter 2 can be used by multiple users, and the storage means 15 can be configured to retrievably store multiple user configurations, i.e., the reference breath value for an individual user.

FIG. 3 shows a third of embodiment of the calorimeter 3.

The calorimeter 3 comprises the features of the first embodiment and an input means 16. The input means 16 is provided, for example, for a user to enter user parameters such as their height, weight, age and gender. The constant OC is also influenced by these factors, just as metabolism in general. The user parameters are used by the processing means to more accurately determine a user's metabolic rate.

The input means 16 is provided on the housing. Alternatively, the input means 16 can be provided on an external apparatus such as a computer. The input means can be, for example, a key pad suitable for the user to input user parameters.

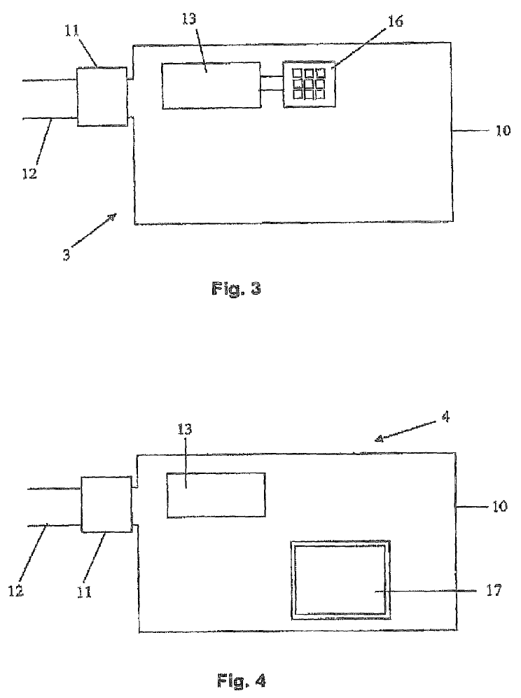

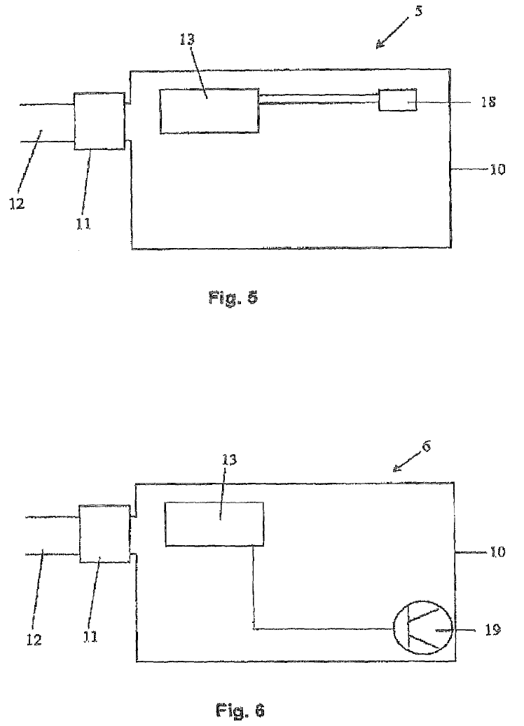

FIG. 4 shows a fourth embodiment of the calorimeter 4. The calorimeter 4 comprises the features of the first embodiment and a display 17. The display 17 displays, for example, a user parameter and a metabolic rate measurement.

The display 17 is a liquid crystal display provided on the housing. However, the display could be any suitable display means. For example, the display 17 could be provided using a personal computer. In another example, the display 17 could be a printer.

FIG. 5 shows a fifth embodiment of the calorimeter 5. The calorimeter 5 comprises the features of the first embodiment and a timing means 18. The timing means 18 is provided to time the amount of time a user has been in fluid communication with the calorimeter 5.

The amount of time a user has been in fluid communication with the calorimeter or breath time can be used to aid the calculation of the volume of a breath. For example, if the volume sensor 11 is a flow sensor, the flow sensor communicates fluctuations in a velocity of a breath to the processing means 13, whilst the timing means 18 determines a breath time. The processing means 13 then determines an average velocity (AvVel) for the breath and uses the breath time in conjunction with the area of the fluid inlet 12 to calculate the volume of a breath (VOB):

VOB=(AvVel*area of fluid inlet)/breath time

The timing means 18 can also be used to measure a reference breath period. In this case, the timing means 18 can also be used to stop the calorimeter 5 recording a reference breath at the end of the reference breath period. The reference breath can be any suitable period of time. For example, the reference breath period can be a period of five minutes.

The processing means 13 could comprise the timings means 19.

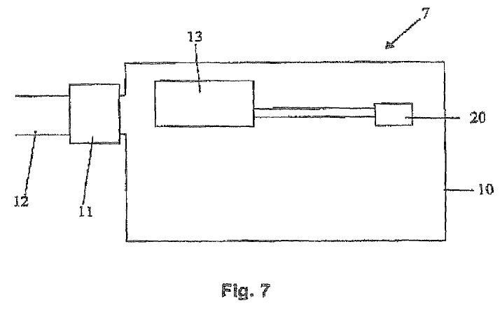

FIG. 6 shows the sixth embodiment of the calorimeter 6. The calorimeter 6 comprises the features of the first embodiment and a warning means 19. The warning means 19 issues a signal when a reference breath period has finished. The warning means 19 also issues a signal when a predetermined number of breaths have been delivered to the calorimeter 6.

The warning means 19 comprises any suitable means or combination of means. For example, the warning means 19 could be an audible signal, a visual signal or a tactile signal.

FIG. 7 shows the seventh embodiment of the calorimeter 7. The calorimeter 7 comprises the features of the first embodiment and a breath counter 20. The breath counter 20 communicates a breath count to the processing means 13. The breath count is used by the processing means 13 to determine when a user has provided the correct number of breaths or the total number of breaths in a period of time. The processing means 13 then provides the user with their metabolic rate reading.

The processing means 13 can use the breath counter 20 to determine the average volume of a user's breath. For example, if the breath counter 20 detects three breaths, the average volume of breath (VOB) will be as follows:

VOB=(VOB1+VOB2+VOB3)/3

The processing means 13 can provide the breath counter 20 using the volume sensor 11. For example, when the volume sensor 11 senses a breath, the processing means 13 updates the breath counter 20.

Alternatively, the breath counter 20 can be a distinct sensor, such as a pressure sensor, and be self-updating.

The first through seventh embodiments are merely exemplary and other embodiments are of course possible. Such embodiments can be made by combining any of the features of the first through seventh embodiments. For example, the fifth embodiment can be combined with the sixth embodiment and the warning means 19 configured to provide a signal to a user when the reference period has ended.

Another possible combination is to combine the second and sixth embodiments. In such a combination where the device is configured for multiple users, the warning means can be used, for example, to warn a user when they have selected an incorrect user configuration.

FIG. 1 shows a calorimeter 1 in which the housing 10 contains the processing means 13. However, other embodiments are of course possible where the processing means 13 is provided partially on a computer and in the housing 10. For example, the processing means 13 in the housing 10 could be provided to calculate the number of calories based on the volume sensor 11 measurement and the processing means 13 on the computer is then used to adjust this value with, for example, the user parameters to calculate the user's metabolic rate. Alternatively, the processing means 13 could be provided on a computer and the volume sensor 11 communicates directly with the computer.

Each calorimeter 1-7 is handheld. However, the calorimeter can also be desk mountable. The desk mountable calorimeter comprises a flexible fluid inlet 12.

Each calorimeter 1-7 can also comprise other sensors to improve its accuracy. The other sensors include humidity sensors, temperature sensors, altitude sensors, etc.

Each calorimeter 1-7 is less complex than other calorimeters. Accordingly, each calorimeter 1-7 can be manufactured efficiently, is easily calibrated and easy to use.

Each calorimeter 1-7 can be used to provide a metabolic rate reading that provides a sufficiently accurate indication of a user's metabolic rate. Each calorimeter 1-7 can operate without any means to measure or determine the chemical constituents used, consumed or produced by a user. Each calorimeter 1-7 can operate with means to measure or determine any physical characteristics, such as a heart rate, of the user.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A calorimeter comprising:
a volume sensor to measure a volume of a breath, and
a processing means to calculate a metabolic rate from the output of the volume sensor; and wherein
the calorimeter calculates a metabolic rate without means to measure the chemical constituents present in a breath.

2. The processing means according to claim 1, wherein the processing means uses a constant OC representing the oxygen content of a breath to calculate a metabolic rate.

3. The processing means according to claim 2, wherein the processing means OC is in a range of 6 to 8.

4. The processing means according to claim 3, wherein the processing means, OC is 6.961.

5. The processing means according to claim 1, wherein the processing means is configured to calculate a metabolic rate by multiplying OC by the volume of a breath.

6. The processing means according to claim 1, wherein the calorimeter determines a metabolic rate only using an output from the volume sensor.

7. The processing means according to claim 1, wherein the volume sensor measures the volume of at least one breath.

8. The processing means according to claim 7, wherein the processing means calculates a metabolic rate using the volume of at least one breath.

9. The processing means according to claim 1, wherein the volume sensor measures a volume of a plurality of breaths.

10. The processing means according to claim 9, wherein the processing means calculates a metabolic rate by averaging a volume of a plurality of breaths.

11. The processing means according to claim 1, wherein the calorimeter is configured to determine a metabolic rate using at least one reference breath.

12. The processing means according to claim 1, wherein the volume sensor is a pressure sensor.

13. The processing means according to claim 1, wherein the volume sensor is a flow sensor.

14. The processing means according to claim 1, wherein the calorimeter comprises a housing.

15. The processing means according to claim 14, wherein the volume sensor is located in the housing.

16. The processing means according to claim 14, wherein the housing comprises a fluid inlet arranged in fluid communication with the volume sensor.

17. The processing means according to claim 16, wherein the fluid inlet is an opening on the housing.

18. The processing means according to claim 16, wherein the fluid inlet is a tube extending from the housing.

19. The processing means according to claim 16, wherein the fluid inlet comprises a mouthpiece.

20. The processing means according to claim 19, wherein the mouthpiece is separable from the fluid inlet.

21. The processing means according to claim 1, wherein the calorimeter further comprises a breath counter.

22. The processing means according to claim 21, wherein the breath counter communicates a breath count to the processing means.

23. The processing means according to claim 22, wherein the processing means uses the breath count in the metabolic rate calculation.

24. The processing means according to claim 21, wherein the breath counter is configured to use the volume sensor.

25. The processing means according to claim 21, wherein the breath counter uses a breath count sensor.

26. The processing means according to claim 1, wherein the calorimeter further comprises a timing means.

27. The processing means according to claim 26, wherein the timing means communicates a breath time to the processing means.

28. The processing means according to claim 27, wherein the breath time is used in the metabolic rate calculation.

29. The processing means according to claim 28, wherein the breath time is used when calculating the volume of a breath.

30. The processing means according to claim 26, wherein the timing means is located in the housing.

31. The processing means according to claim 1, wherein the calorimeter further comprises an input means to receive at least one user parameter.

32. The processing means according to claim 31, wherein the input means communicates the at least one user parameter to the processing means.

33. The processing means according to claim 32, wherein the at least one user parameter is used in the metabolic rate calculation.

34. The processing means according to claim 1, wherein the calorimeter further comprises a display to display at least a metabolic rate.

35. The processing means according to claim 1, wherein the calorimeter further comprises a warning means to provide a warning signal to the user.

36. The processing means according to claim 35, wherein the warning signal is provided to indicate a reference breath period has ended.

37. The processing means according to claim 35, wherein the warning signal is provided to indicate that a required number of breaths have been received.

38. The processing means according to claim 1, wherein the calorimeter further comprises a storage means to store the volume sensor measurement.

39. The processing means according to claim 38, wherein the storage means stores a user configuration.

40. The processing means according to claim 38, wherein the storage means stores a plurality of user configurations.

41. The processing means according to claim 41, wherein the calorimeter can be configured based on an individual user configuration retrieved from the plurality of user configurations.

42. A method of calculating a metabolic rate, the method comprising the steps of:
(a) providing a calorimeter comprising a volume sensor and a processing means,
(b) receiving at least one breath from a user,
(c) determining the volume of the at least one breath,
(d) calculating the metabolic rate of the user without measuring the chemical constituents present in a breath.

43. The processing means according to claim 42, wherein the step (d) comprises calculating the metabolic rate using a constant OC representing the oxygen content of a breath.

44. The processing means according to claim 42, wherein the step (d) comprises calculating the metabolic rate by multiplying the volume of at least one breath by OC.

45. The processing means according to claim 42, wherein the step (a) further comprises providing a timing means and step (c) further comprises providing a breath time and calculating the volume of at least one breath using the breath time.

46. The processing means according to claim 42, wherein the step (a) further comprises providing a breath counter and step (c) further comprises determining the volume of a plurality of breaths.

47. The processing means according to claim 46, wherein the step (c) comprises calculating the average volume of the plurality of breaths.

48. The processing means according to claim 46, wherein the step (c) comprises receiving a breath count before calculating the average volume of the plurality of breaths.

49. The processing means according to claim 42, wherein the step (c) comprises receiving a plurality of breathing times before calculating the average volume of the plurality of breaths.

50. The processing means according to claim 42, wherein the method further comprises step (e) displaying the metabolic rate.
